(19) United States
(12) Patent Application Publication
(10) Pub. No.: US 2023/0285536 A1
(43) Pub. Date: Sep. 14, 2023

(54) EDIBLE CELL BASED VACCINES

(71) Applicant: Trustees of Tufts College, Medford, MA (US)

(72) Inventors: David Kaplan, Concord, MA (US); Natalie Rubio, Medford, MA (US); Andrew Stout, Cambridge, MA (US)

(21) Appl. No.: 18/002,899

(22) PCT Filed: Jun. 24, 2021
(86) PCT No.: PCT/US2021/070768

§ 371 Date: Dec. 22, 2022

Related U.S. Application Data
(60) Provisional application No. 63/043,635, filed on Jun. 24, 2020.

(51) Int. Cl. A61K 39/145; C12N 5/077; C12N 5/07; C07K 14/11; A61P 31/16

(52) U.S. Cl. CPC A61K 39/145; C12N 5/0659; C12N 5/0601; C07K 14/11; A61P 31/16; A61K 2039/5156

(57) ABSTRACT

The present invention provides animal cells that have been engineered to express one or more viral antigens. The engineered cells can be formulated into cultured meat products that can be used as edible vaccines.

### CROSS-REFERENCE TO RELATED APPLICATIONS

This application claims benefit to U.S. Provisional Application No. 63/043,635 filed on Jun. 24, 2020, the contents of which are incorporated by reference in its entirety.

### STATEMENT REGARDING FEDERALLY SPONSORED RESEARCH

This invention was made with government support under grant P41EB027062 awarded by the National Institutes of Health. The government has certain rights in the invention.

### SEQUENCE LISTING

A Sequence Listing accompanies this application and is submitted as an ASCII text file of the sequence listing named “166118_01054_ST25.txt” which is 216 KB in size and was created on Jun. 10, 2021. The sequence listing is electronically submitted via EFS-Web with the application and is incorporated herein by reference in its entirety.

### BACKGROUND

Vaccination is the preferred mode of preventing infectious disease. Safe and effective vaccines have been employed for over 60 years and have reduced the number of deaths caused by infectious diseases by more than 95% (Bonanni et al., 2015). Despite this success, infectious diseases remain a leading cause of death globally. Researchers estimate that 20% of infectious disease-related deaths could be prevented by currently available vaccines (World Health Organization, 2015). For example, the global mortality rate for the flu is between 290K and 650K deaths per year. Yet, despite the widespread availability of the flu vaccine in the United States, the 2017-2018 flu season resulted in up to 45 million illnesses, 810 million hospitalizations, and 61,000 deaths (Centers for Disease Control and Prevention, 2020). A significant portion of these cases are thought to be due to the limited number of people who receive seasonal flu vaccinations. For instance, for the 2018-2019 flu season, 41% of Americans reported they did not intend to get a flu vaccine.

Current vaccine production techniques are plagued by numerous limitations, including lengthy production times, distribution limits, and suboptimal routes of administration. For example, each year, it takes approximately 7 months to develop egg-based and cell-based seasonal influenza vaccines and approximately 5 months to develop recombinant-based vaccines (Chen et al., 2020). Further, the ideal storage temperature for conventional influenza vaccines is 5° C. and temperature fluctuations can reduce antigen efficacy. Thus, to reduce the immunization coverage gap in developing countries, the range of allowable temperatures for vaccine storage and distribution must be widened (Ashok et al., 2017). Finally, the seasonal influenza vaccine is predominantly administered via an intramuscular injection, despite the fact that many Americans (28%) avoid receiving a flu shot due to a fear of needles (Young, 2018). Furthermore, oral or nasal administration is superior to intramuscular administration because it stimulates both mucosal (i.e., IgA antibodies) and systemic (i.e., IgG antibodies) immunity.

When oral administration of vaccines is possible, it is often preferred due to improved safety and convenience. However, patient compliance can also be an issue for conventional oral administration of drugs, as 10-20% of the population reports difficulty swallowing pills (Schiele et al., 2013). Thus, there is growing interest in edible vaccines, i.e., vaccines that are provided as a food formulation. To date, edible vaccines have been explored primarily in the form of plant-based vaccines, in which consumable plants are engineered to produce antigens against infectious agents. The benefits of edible vaccines include less stringent storage requirements, increased patient compliance, and reduced production, administration, and distribution costs. Further, edible vaccines have reduced side effects due to lack of pathogens, toxins, and allergens (e.g., from eggs) that are commonly present in traditional vaccines. While their benefits are noteworthy, edible plant-based vaccines have several limitation, including variability in antigen concentration, differences in plant versus human glycosylation patterns, and a lack of comprehensive research.

Thus, there is a need in the art for improved oral vaccination platforms and edible vaccines.

### SUMMARY OF THE INVENTION

In a first aspect, the present invention provides an engineered cell comprising a heterologous polynucleotide encoding a viral antigen. The cell may be a muscle cell, a muscle precursor cell, a fibroblast, or an adipocyte from a non-human mammal, an insect, or a fish.

In a second aspect, the present invention provides a cultured meat product comprising comprising an engineered cell described herein seeded on a food safe substrate. In some aspects, the meat product comprises a confluent serum-free culture of the engineered cells.

In a third aspect, the present invention provides an edible vaccine comprising a cultured meat product described herein or an engineered cell described herein.

In a fourth aspect, the present invention provides a method for inducing an immune response against a virus in a subject. The method comprises administering an effective amount of an edible vaccine described herein to the subject.

### DETAILED DESCRIPTION OF THE INVENTION

The present invention provides cells that have been engineered to express one or more viral antigens. The engineered cells can be formulated into cultured meat products that can be used as edible vaccines.

Recent advances in in vitro meat production provide an opportunity to develop new edible vaccine formulations, in which antigens are expressed in cells that are combined to form a cultured meat product. These cultured meat-based edible vaccines may offer advantages over plant-based edible vaccines in terms of improved dose control and consistency, improved bioavailability and drug activity, improve glycosylation patterns, and reduced susceptibility to natural factors. Importantly, cultured meat-based edible vaccines also offer several significant advantages over conventional vaccines. For example, edible vaccines (1) can be produced rapidly, i.e., in 3-4 months as compared to the 7 months required to produce egg-based and cell-based vaccines; (2) have a widened range of allowable storage temperatures due to the stabilization of antigens within cells/tissues up until the point of delivery; (3) stimulate both mucosal and systemic immunity; and (4) circumvent issues with patient needle/pill compliance.

### Engineered Cells:

In a first aspect, the present invention provides an engineered cell comprising a heterologous polynucleotide encoding a viral antigen. The cell may be a muscle cell, a muscle precursor cell, a fibroblast, or an adipocyte from a non-human mammal, an insect, a fish, or a bird.

As used herein, the term “engineered cell” refers to a cell that comprises an exogenous polynucleotide. The cells of the present invention may be generated by introducing one or more heterologous polynucleotides encoding a viral antigen into the cell to produce a genetically engineered cell. The cell may be engineered to transiently or stably express the viral antigen. The heterologous polynucleotide encoding the viral antigen may be introduced into the cell using any transfection, transformation, or transduction method known in the art (e.g., via microinjection, encapsulation, or electroporation). In some embodiments, the polynucleotide is integrated into the genome of the cell. For example, the polynucleotide may be integrated into the genome using a zinc-finger nuclease, TALENs, CRISPR/Cas mediated genome editing, or a transposon (e.g., Sleeping Beauty, PiggyBac, Tol1, Tol2, Minos, Frog Prince, Passport, Hsmarl, or Harbinger3_DR). Other suitable methods may be used that are well known and understood in the art.

As used herein, the terms “polynucleotide” and “nucleic acid” are used interchangeably to refer to a polymer of DNA or RNA, which may be of natural or synthetic origin. The polynucleotides of the present invention are “heterologous”, meaning they are not naturally associated with the host cell into which they have been introduced.

The polynucleotides may be provided to the cell in the form of a construct or a vector (i.e., a construct or vector comprising a polynucleotide described herein). As used herein, the term “construct” refers to an artificially constructed polynucleotide. Constructs may be entirely synthetic or may be recombinant polynucleotides that comprise polynucleotide sequences derived from at least two different natural sources. The term “vector” refers to a nucleic acid molecule that is capable of propagating another nucleic acid to which it is linked. The term includes the vector as a self-replicating nucleic acid structure as well as the vector incorporated into the genome of a host cell into which it has been introduced. Vectors suitable for use with the present invention comprise a polynucleotide encoding a viral antigen described herein and additional heterogeneous sequence necessary for proper propagation of the vector and expression of the encoded antigen. Suitable vectors include, for example, episomal vectors, viral vectors (e.g., retrovirus, adenovirus, baculovirus), plasmids, and RNA vectors.

The construct or vector used with the present invention may include a promoter that is operably linked to one or more polynucleotides described herein. As used herein, the term “promoter” refers to a DNA sequence capable of controlling the expression of a downstream coding sequence. Promoters may be derived in their entirety from a native gene, or be composed of different elements derived from different promoters found in nature, or even comprise synthetic DNA segments. The promoter used with the present invention may be a heterologous promoter (i.e., a promoter that is not naturally associated with the host cell genome) or an endogenous promoter. Suitable promoters for use with the present invention include, but are not limited to, constitutive, inducible, temporally regulated, developmentally regulated, chemically regulated, tissue-preferred, and tissue-specific promoters. The promoter may be a plant, animal, bacterial, fungal, or synthetic promoter. Promoters that are commonly used in mammalian cells include, without limitation, promoters for Rous sarcoma virus (RSV), human immunodeficiency virus (HIV-1), cytomegalovirus (CMV), Dox-inducible promoter (e.g., Tet Response Element (TRE)), ubiquitin C (Ubc), CMV early enhancer/chicken beta actin (CAG), human beta actin, phosphoglycerate kinase 1 (PGK1), SV40 virus, and the like. Promoters that are commonly used in insect cells include upstream activating sequence (UAS), actin 5c (Ac5), and polyhedrin. Promoters that are commonly used in fish cells include Xenopus laevis elongation factor 1α promoter (XlEef1a1) and ocean pout antifreeze protein promoter (OP5a). Other suitable promoters include the T3, T7 and SP6 promoter sequences, which are often used for in vitro transcription of RNA.

In some embodiments, the construct or vector used with the present invention encodes a reporter protein. A “reporter protein” is a protein that confers to a cell a characteristic that is easily identified or measured. A reporter protein is often used to indicate whether a certain gene has been taken up by or expressed in the cell. Suitable reporter proteins include, for example, proteins that can produce a fluorescent signal, luminescent signal, colorimetric signal, wavelength absorbance, or radioactive signal. Exemplary reporter proteins include β-glucuronidase (GUS), β-lactamase, luciferase, α-amylase, green fluorescence protein, and α-galactosidase, among others. In some embodiments, the reporter protein is a selectable marker. As used herein, the term “selectable marker” refers to a protein that protects a cell from a selection agent that would normally kill it or prevent its growth or a protein that allows for identification of the cell in which it is produced. A selectable marker may be a compound that confers resistance to an otherwise toxic compound. For example, in some embodiments, the selectable marker confers resistance to an antibiotic. Cells that have been transfected with a construct encoding a selectable marker can be exposed to the selection agent to select for cells comprising the construct. In another embodiment, the selectable marker may be a fluorescent protein.

As used herein, the term “antigen” refers to a molecule that can initiate an immune response (i.e., a humoral and/or a cellular immune response) in a recipient. Antigens can be any type of biologic molecule including, for example, simple intermediary metabolites, sugars, lipids, and hormones as well as macromolecules such as complex carbohydrates, phospholipids, nucleic acids, and proteins. The antigens used with the present invention are “viral antigens”, meaning that they are virus-specific antigens that elicit an immune response against a virus in a recipient. Typically, a viral antigen is present on the surface of the virus and triggers an immune response in the recipient by reacting with or binding to one or more immune cells.

The viral antigen used with the present invention may be from any virus of interest. For example, in some embodiments, the viral antigen is from an influenza, a coronavirus, hepatitis B, a human papilloma virus, a parvovirus, or a Norwalk virus, among others. Suitable antigens derived these viruses include, for example, an influenza hemagglutinin protein, an influenza neuraminidase protein, an influenza matrix protein, a coronavirus spike protein, a coronavirus envelope protein, a coronavirus membrane protein, a coronavirus receptor binding domain protein, a major hepatitis B surface antigen protein, a human papillomavirus L1 protein, a parvovirus viral protein, and a Norwalk virus capsid protein. A more detailed description of these viral antigens is provided in Example 2, and exemplary amino acid sequences of these antigens are provided as SEQ ID NOs:1-50. The polynucleotides of the present invention may encode one or more of these exemplary antigens or an immunogenic fragment thereof.

Influenza vaccines are commonly formulated against multiple influenza subtypes. For example, three influenza subtypes (i.e., H₁N₁, H₃N₂, or H₅N₁) are combined to create a trivalent influenza vaccine. Thus, in some embodiments, the cell is engineered to express a combination of HA proteins from the H₁N₁, H₃N₂, and H₅N₁ influenza subtypes. In some embodiments, the viral antigen has a sequence that is at least 85%, 90%, 95%, 98%, 99%, or 99.9% identical to SEQ ID NO:1 (i.e., HA type 1), SEQ ID NO:3 (i.e., HA type 3), and/or SEQ ID NO:5 (i.e., HA type 5).

“Percentage of sequence similarity” or “percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

In some embodiments, the cell is engineered to express a virus-like particle (VLP). As used herein, a “virus-like particle (VLP)” is a particle that includes one or more viral proteins and mimics at least a portion of the structure of the native virus but lacks the viral genome. The VLP may include any combination of the viral antigen proteins as described herein. In some embodiments, the VLP includes at least one influenza HA protein. In some embodiments, the VLP includes at least two influenza HA proteins. In some embodiments, the VLP includes at least three influenza HA proteins. Preferably, the one or more HA proteins are for variant strains of influenza, and thus will confer an immune response to multiple strains of influenza within one VLP.

In some embodiments, the cell is engineered to express the viral antigen as a fusion protein with a protein adjuvant. As used herein, the term “adjuvant” refers to a substance that enhances the body's immune response to an antigen. Suitable protein adjuvants may include, but are not limited to, interleukin 12 (IL-12), cucumber mosaic virus tetanus toxoid (CMV-TT), and bacterial toxins (Cholera toxin, heat labile enterotoxin), among others. One skilled in the art is able to determine suitable protein adjuvants for use in the present invention.

The engineered cell of the present invention may be any animal cell that can be stably maintained, grown, and expanded in culture. Suitable animal cells for use with the present invention include, mammalian cells, insect cells, fish cells, and avian cells. Suitable mammalian cells include porcine, bovine, cervine, hircine, leporine, and murine cells. In some embodiments, the cell is from a cow of the species Bos taurus. For use in a cultured meat product, as described below, the mammalian cell is a non-human mammalian cell.

Methods for use of genetically engineered cells in a cultured meat product are described, for example, in Publication No. WO2020243695 of International Application No. PCT/US2020/035526, which is incorporated herein by reference in its entirety. Methods for the use of insect cells, specifically, in a cultured meat product are described in Publication No. WO2020131661 of International Application No. PCT/US2019/066452, which is incorporated herein by reference in its entirety. The present engineered cell in the cultured meat product designed herein may as described in either of these publications.

The animal cells of the present invention may be any type of cell including, for example, a primary cell, an immortalized cell, or a cell differentiated from a pluripotent cell (e.g., an embryonic stem cell, induced pluripotent cell, etc.). Primary cells may be grown and proliferated in culture for 1, 2, 3, 4, 5, 6, or more passages prior to use. In some embodiments, the cell is an immortalized cell that expresses TERT and CDK4 and has been spontaneously immortalized through continuous culture, or engineered to express other immortalization genes.

In some embodiments, the cell is a mammalian cell. In some embodiments, the mammalian cell is muscle precursor cell. For example, in some embodiments, the cell is a mammalian muscle precursor cell that expresses paired box protein 7 (Pax7). In some embodiments, the cell is a muscle precursor satellite cell that expresses Pax7. In some embodiments, the cell is a Pax7+ bovine satellite cell.

In other embodiments, the mammalian cell is a muscle cell. Mammalian muscle cells can be characterized by the expression of actin, myogenin, and myosin heavy chain (MHC) and the formation of multinucleated myotubes. For example, in some embodiments, the cell is a MHC⁺ actin⁺ mammalian multinucleated myotube. In some embodiments, the myotube is produced by differentiation from mammalian satellite cells. To differentiate the satellite cells into myotube cells, the satellite cells are cultured to confluency in any culture medium that supports their growth (e.g., B8 medium or DMEM+20% fetal bovine serum). Then, the confluent cells are differentiated in culture medium that includes a Neurobasal/L15 (1:1) basal media supplemented with epidermal growth factor (EGF; 0.01-5 ng/ml; preferably 0.5 ng/mL), insulin-like growth factor 1 (IGF-1; 0.01-5 ng/ml; preferably 0.05 ng/mL), and 1% Antibiotic-Antimycotic. In some embodiments, the cells are differentiated for 3-30 days.

In other embodiments, the mammalian cell is an adipocyte. Mammalian adipose cells are characterized by expression of peroxisome proliferator-activated receptor gamma (PPARγ) and increased lipid production. In some embodiments, the adipocyte is produced by differentiation from mammalian satellite cells. Lipid accumulation can be initiated in satellite cells using a differentiation media containing free fatty acids (FFAs). Specifically, a 3-FFA cocktail of linoleic acid, erucic acid, and elaidic acid at equal concentrations (e.g., between about 10 uM and about 500 uM) induces accumulation of intracellular lipids over about six days in culture. A more detailed description of adipogenic differentiation from mammalian satellite cell is provided in Fish et al. (Trends Food Sci Technol., 2020, 98:53-67), which is incorporated by reference herein.

In other embodiments, the cell in an insect cell. In some embodiments, the insect cell is a muscle cell or a muscle precursor cell (i.e., a muscle progenitor-like cell). Insect muscle cells are long, multinucleated myotubes that can be identified by expression of myosin heavy chain and ecdysone receptor (EcdR). In some embodiments, insect muscle cells also express connectin and/or neuroglian. Insect muscle cells can also be characterized by spontaneous cell contraction or by contraction following stimulation with extracellular potassium. Additionally, insect muscle cells can be characterized by growth and survival in the prolonged absence of medium refreshment. For example, the insect muscle cells can survive and grow for at least about 2 days, at least about 5 days, at least about 10 days, at least about 15 days, at least about 20 days, or at least about 25 days when the medium is not changed or supplemented. In some embodiments, the insect muscle cells are primary Drosophila muscle cells. In some embodiments, the insect muscle cells are primary cells from Manduca sexta embryos or adult muscles. In some embodiments, the insect muscle cells are derived from Manduca sexta embryonic precursor cells (Ms-EPC). In some embodiments, the insect muscle precursor cells are Drosophila melanogaster adult muscle progenitor-like cells (DrAMPCs).

In some embodiments, the insect muscle cell is produced by differentiation of an insect muscle progenitor-like cell. This is accomplished by culturing the insect muscle progenitor-like cells in serum-free culture medium including insect molting hormone 20-hydroxyecdysone (20-HE) for a time under suitable conditions for cells to elongate and fuse (i.e., differentiate). In some embodiments, the insect muscle progenitor-like cells are cultured while adhered to a substrate in serum-free culture medium including 20-HE. Insect muscle progenitor-like cell are cultured in serum-free culture medium supplemented with 20-HE for at least about 0.5 days, at least about 1 day, at least about 2 days, at least about 3 days, or at least about 5 days to form insect muscle cells expressing myosin heavy chain and EcdR. In some embodiments, differentiation of insect muscle cells is observed after about 24 hours in culture with 20-HE. 20-HE is present in the culture medium at a concentration of at least 20 ng/ml, at least about 30 ng/ml, at least about 40 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 mg/ml, at least about 900 ng/ml, or at least about 100 ng/ml. The 20-HE may be present in the culture medium at a concentration of between about 20 ng/ml and about 2000 ng/ml, between about 40 ng/ml and about 1500 ng/ml, between about 50 ng/ml and about 1000 ng/ml, between about 100 ng/ml and about 1000 ng/ml, or between about 100 ng/ml and about 500 ng/ml. In some embodiments, the serum-free culture medium is a commercially available culture medium such as Ex-Cell 405. In some embodiments, the insect muscle progenitor-like cells are adhered to a substrate in the presence of serum-free culture medium then supplemented with 20-HE. In some embodiments, the insect muscle progenitor-like cells are cultured in serum-free medium supplemented with 20-HE while adhered to a substrate coated with Concanavalin A, laminin, and/or poly-lysine. In some embodiments, the culture additionally includes methoprene (JH). In some embodiments, the culture additionally includes sericin protein. In some embodiments, the culture additionally comprises an anti-agglomeration agent, such as dextran sulfate.

### Cultured Meat:

In a second aspect, the present invention provides a cultured meat product comprising a confluent, serum-free culture comprising an engineered cell described herein seeded on a food safe substrate.

As used herein, a “cultured meat product” is an edible meat product produced from cell culture rather than a whole organism. Ideally, a cultured meat product is visually identical to farmed meat and is palatable to consumers. Methods for producing cultured meat are known and described in the art. See, for example, Post (Meat Science, 2012, 92, 297-301), Warner (Animal, 2019, 13(12):3041-3058), U.S. Pat. Nos. 6,835,390, and 7,270,829.

The cultured meat products of the present invention include engineered cells described herein seeded on a food safe substrate and cultured to confluence. As used herein, “confluent” refers to cells in tissue culture that form a cohesive single cell layer that covers the substrate. The cells are seeded on the substrate at a density between about 20,000 cell/cm² and about 400,000 cell/cm², between about 30,000 cells/cm² and about 350,000 cells/cm², or between about 35,000 cells/cm² and about 300,000 cells/cm². In some embodiments, the cells are seeded at a density of about 50,000 cells/cm². In some embodiments, the cells are seeded at a density of about 40,000 cells/cm², about 50,000 cells/cm², about 60,000 cells/cm², about 70,000 cells/cm², about 80,000 cells/cm², about 100,000 cells/cm², about 150,000 cells/cm², about 200,000 cells/cm², about 250,000 cells/cm², about 300,000 cells/cm² or about 350,000 cells/cm². In some embodiments, the cells become non-adherent on the food safe substrate once they reach confluence and lift off the food safe substrate without enzymatic dissociation.

In addition to the engineered cells described herein, the cultured meat products may include the plant based proteins and unmodified (i.e., wild-type) plant or animal cells. In some embodiments, the engineered cells described herein make up about 1% to about 100% of the cultured meat product based on weight or based on cell count.

As used herein, “serum-free” refers to culture conditions that do not contain serum or serum replacement, or that contain essentially no serum or serum replacement. For example, an essentially serum-free medium can contain less than about 1%, 0.9%, 0.8%, 0.7%, 0.6%, 0.5%, 0.4%, 0.3%, 0.2%, or 0.1% serum. As used herein, “serum-replacement,” refers to an animal serum or animal product-based replacements for the serum traditionally used in culture medium.

In some embodiments, the culture medium may include a plant-based or yeast-based extract in place of serum. Exemplary yeast and plant-based serum alternatives include hydrolyzed proteins from soy, yeast, wheat gluten, cottonseed, or corn, as well as rapeseed peptide fractions, maitake mushroom extract, and silk derived sericin protein. Plant-based and yeast-based serum alternatives do not fall under the definition of “serum-replacement”. In other words, a culture medium that comprises a plant-based or yeast-based serum alternative can be considered serum-free.

One example of a serum-free medium that can be used with the present invention is B8. B8 medium includes DMEM:F12 (1:1) basal medium supplemented with L-ascorbic acid 2-phosphate (0.1 ug/ml to 500 ug/ml; preferably about 200 ug/mL), insulin (0.1 ug/ml to 100 up/ml; preferably about 20 ug/mL), transferrin (0.1 ug/ml to 100 ug/ml; preferably about 20 ug/mL), sodium selenite (0.1 ug/ml to 100 ug/ml; preferably 20 ng/mL), FGF-2 (0.01 ug/ml to 100 ug/ml; preferably 10 ng/mL), neuregulin 1 (NRG-1; 0.001 ng/ml to 50 ng/ml; preferably 0.1 ng/mL), and TGFβ-3 (0.001 ng/ml to 50 ng/ml; preferably 0.1 ng/uL). In some embodiments, B8 medium additionally includes insulin-like growth factor 1 (IGF-1; about 10 ng/mL).

In some embodiments, the medium used in the cultured meat product is a defined medium. The terms “defined culture medium” and “defined medium” indicate that the identity and quantity of each medium ingredient is known.

In some embodiments, the culture medium includes, cinnamon, monolaurin (a derivative of coconut lauric acid), honey, or combinations thereof in addition to or instead of an antibiotic component.

Culturing can take place in any appropriate vessel (e.g., in two-dimensional plates or three-dimensional shaker flask culture). In some embodiments, the cells are cultured in shaker flasks. In some embodiments, the cells are cultured in a static suspension culture (e.g., in ultra-low attachment plates).

To form the cultured meat products of the present invention, engineered cells are seeded on a food safe substrate. As used herein, the term “food safe substrate” refers to substrates that are edible or are safe for human consumption. Such substrates are necessary in case a portion of the substrate remains affixed to the cultured meat product when it is consumed. Suitable food safe substrates are known in the art and include, but are not limited to, chitosan substrates, cellulosic substrates, silk substrates, alginate substrates, starch substrates, textured vegetable protein substrate, mycelium substrates, and whey substrates.

In some embodiments, the substrate is a chitosan substrate. Chitosan may be derived from the chitin of organisms including, but not limited to, mushrooms, crustaceans, insects, green algae, and yeast. In some embodiments, the food safe substrate is a mushroom-chitosan substrate. The chitosan substrate can be tuned to change the adherence and growth of the mammalian muscle cell culture. For example, increasing the chitosan concentration in the substrate generally decreases adhesion of the mammalian muscle cells. The concentration of chitosan in the food safe substrate of the cultured meat product can be 1%, 2%, 3%, 4%, 5%, 6%, 7%, 8%, 9%, 10%, 12%, or 14%. In some embodiments, the concentration of chitosan is between about 1% and about 8%. In some embodiments, the concentration of chitosan is between about 2% and about 6%. In some embodiments, the concentration of chitosan is at least about 1%, at least about 2%, at least about 3%, at least about 4%, at least about 5%, at least about 6%, at least about 7%, or at least about 8%.

In other embodiments, the food safe substrate is a cellulose-based substrate, such as a substrate formed from decellularized plants (e.g., decellularized spinach or apples).

In some embodiments, the food safe substrate is in the form of a two-dimensional film. In other embodiments, the food safe substrate is in the form of a three-dimensional matrix or sponge, and the mammalian muscle cells form continuous muscle fibers when cultured in the matrix or sponge substrate.

Three-dimensional chitosan substrates or sponges suitable for use in the formation of muscle fibers and cultured meat products described herein can be produced using methods known in the art. For example, chitosan sponges can be formed using directional freezing. In this method, chitosan is dissolved in a solvent (e.g., acetic acid) and the chitosan solution is poured into tubes. One end of the tubes is exposed to a freezing agent (e.g., liquid nitrogen or a slurry of dry ice and ethanol) until the entire solution is frozen. The frozen chitosan is then lyophilized to form the chitosan sponge. The mechanical properties of the chitosan sponge can be tuned by altering the chitosan concentration. Sponges formed using a low concentration chitosan solution (e.g., 1%, 2%, 3% chitosan) have a lower elastic moduli, whereas sponges formed using a high concentration chitosan solution (e.g., 6%, 7%, 8% chitosan) have higher elastic moduli.

### Edible Vaccines:

In a third aspect, the present invention provides an edible vaccine comprising a cultured meat product described herein or an engineered cell described herein.

As used herein, the term “vaccine” refers to a composition containing an antigen. A vaccine is administered to an individual to stimulate that individual's immune response to said antigen. The vaccines of the present invention are “edible”, meaning that they are safe to eat.

### Methods:

In a fourth aspect, the present invention provides a method for inducing an immune response against a virus in a subject. The method comprises administering an effective amount of an edible vaccine described herein to the subject.

As used herein, the term “administering” refers to any method of providing a pharmaceutical preparation to a subject. Common methods of administering a vaccine include oral administration, subcutaneous administration, intramuscular administration, intradermal administration, and intranasal administration. In preferred embodiments, the edible vaccine is administered orally. The edible vaccines may be administered in a prophylactic manner (i.e., to prevent or ameliorate the effects of a future viral infection) or in a therapeutic manner (i.e., to treat a viral infection).

The terms “effective amount” or “therapeutically effective amount” refer to an amount of a vaccine that is sufficient to induce an immune response in a subject that has received the vaccine. Ideally, the effective amount is sufficient to prevent signs or symptoms of infection by the target virus. Humoral immunity, cell mediated immunity, or both humoral and cell mediated immunity may be induced. The immunogenic response of an animal to a vaccine may be evaluated indirectly, e.g., through measurement of antibody titers or lymphocyte proliferation assays, or directly, e.g., by monitoring signs and symptoms after challenge with the target virus. The protective immunity conferred by a vaccine may be evaluated by measuring a reduction in clinical signs, e.g., the mortality, morbidity, temperature, physical condition, or overall health of the subject. The amount of a vaccine that is therapeutically effective may vary depending on the particular preparation used and the condition of the subject, and may be determined by a physician.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

### EXAMPLES

### Example 1—Edible Vaccines Against Seasonal Influenza

The following example describes a novel vaccine for the seasonal influenza. The vaccines are made using the following steps: (1) edible cells are engineered to produce hemagglutinin (HA) virus-like particles, (2) transfected cells are expanded in a bioreactor to a target density and virus-like particle yield, and (3) cells are harvested and formulated into a food product (e.g., smoothies, minced meat) at the correct dosage.

### Advantages:

The vaccines described in this example combine the benefits of three technologies: (1) recombinant-based vaccines, (2) plant-based edible vaccines, and (3) cell-based food production. The benefits and limitations of each of these technologies is described below.

1. Recombinant-based vaccines—The flu vaccine is generally manufactured in inoculated chicken eggs, which can produce 15 μg of vaccine per 10 mL of fluid (i.e., one egg) (Centers for Disease Control and Prevention, 2019). Three influenza subtypes (A, H1N1; A H3N2, and B) are combined to create a trivalent inactivated influenza vaccine. The flu vaccine is also produced as a cell-based vaccines via infection of mammalian cell cultures. Both of these methods produce a live virus. Alternatively, the flu vaccine can be produced as a subunit recombinant vaccine via genetically engineered cells. Recombinant vaccine production offers increased production speed and reduced allergenicity over egg-based and cell-based vaccines (Vela Ramirez et al., 2017).

The baculovirus-insect expression system is advantageous for recombinant vaccine production due to its simple design and the ability of insect cells to produce post-transcriptional modifications. Flublok, a recombinant flu vaccine produce in insect cells, was approved by the Food and Drug Administration (FDA) in 2013. Flublok is administered as an intramuscular injection. A single dose contains 135 μg of trivalent vaccine (i.e., 45 μg of each of 3 full-length recombinant hemagglutinin proteins). The proteins are expressed from the baculovirus vector in Spodoptera frugiperda (i.e., fall armyworm) cells. The antigens are extracted from the cell culture and purified via chromatography. The cloning, expression and production process takes less than two months. Other key benefits include high antigen yields, high levels of quality control, and comprehensive research. Limitations of recombinant-based vaccines include that they require purification, produce no mucosal immunity, and require administration by healthcare personnel.

2. Edible plant-based vaccines—Oral vaccines can take the form of lyophilized powders that are reconstituted in a single liquid (Vaxchora), multiple liquids (Rotarix, RotaTeq), or provided in capsules (Vivotif). Edible vaccines are also being developed in the form of transgenic plants that are engineered to produce antigens against disease and can be consumed to stimulate mucosal and systemic immunity. For example, vaccines against hepatitis B, Norwalk virus, rabies, and HIV have been expressed in tobacco plants, potatoes, lettuce, tomatoes, and maize (Mishra et al., 2008). In clinical trials, 100 g of raw potato expressing heat-labile enterotoxin B (3.7-15.7 μg/g) were shown to induce immunity. To create an edible vaccine, an antigen that can induce an immune response against the target disease is identified, and a gene encoding the antigen is cloned into a transfer vector, commonly Agrobacterium tumefaciens (Concha et al., 2017). The antigen-encoding gene is integrated into the plant genome and is expressed in the plant tissue. Key benefits of edible plant-based vaccines include combined mucosal and systemic immunity, self-administration, and no time-consuming and costly purification steps. Limitations of these vaccines include variability in antigen concentration, differences in plant versus human glycosylation patterns, and a lack of comprehensive research.

3. Cell-based food—Recently, advances in biotechnology have been applied to the generation of novel foods (e.g., animal-free dairy proteins, recombinant leghemoglobin, cultured meat). Cultured meat is produced by isolating skeletal muscle and adipose-specific stem cells from livestock species, expanding the cells in cultures of xenogen-free growth medium, and differentiating the cells into tissue on food-grade scaffolds (Datar and Betti, 2010). The first proof-of-concept cultured meat was a beef burger constructed from 40 billion bovine muscle cells at Maastricht University in 2013. Today, there are dozens of companies working to commercialize cultured meat. Key benefits of cell-based foods include sustainable production, reduction of foodborne illness, and improved animal welfare (Fraser et al., 2018; Post, 2012). Challenges include achieving large-scale production and reaching price parity with traditional products.

The edible cell-based vaccines described herein are cultured foods that combine many benefits of recombinant-based and plant-based vaccines (e.g., high yields, quality control, dual immunity, self-administration, no purification). By using a cell-based food production approach, we ensure that our production process is safe, sustainable, and humane. Further, use of this technology for a therapeutic application circumvents the scalability and cost obstacles of cell-based food production, as the target market allows for smaller scale production and higher price points than would be required to meet consumer meat demands.

The vaccines described in this example also avoid several key limitations of three technologies: (1) inconsistent dosage, (2) limited efficacy, (3) patient compliance, and (4) cost.

1. Dosage consistency—The edible cell-based vaccines will have therapeutic dosage consistency. Edible plant-based vaccines are currently being developed, but their main limitation is dosage inconsistency. Antigen concentrations vary within and between individual plants and between plant generations. For example, in human clinical trials, potato vectors contained between 3.7-15.7 μg of antigen per gram of potato. In contrast, the cultured meat used in our vaccines will be produced in a similar manner to cell-based vaccines, allowing antigen concentrations to be standardized.

2. Efficacy—The edible cell-based vaccines will stimulate strong immunity. Edible plant-based vaccines comprising virus-like particle (VLP) formulations have demonstrated success in eliciting broad and long-lasting immunity against various pathogens (Mishra et al., 2008). However, humans cannot digest cellulose, which may limit antigen delivery along the gastrointestinal tract. For our cultured meat vectors, bioavailability should be much higher, as VLPs can be engineered to resist degradation until they reach the small intestine and penetrate the mucosal barrier (Schneider-Ohrum and Ross, 2012).

3. Patient compliance—The edible cell-based vaccines will provide pain-free and pill-free administration. Edible vaccines will result in higher patient compliance because they provide an immunization option for populations that have an aversion to needles (20% of adults) or have difficulty swallowing pills (40% of American adults) (Cook, 2016; Schiele et al., 2013). Our cultured meat-based vaccines may also have compliance benefits when compared with existing plant-based vaccines, as cooking and antigen concentration limitations of plant-based vaccines may require patients to eat larger amounts of unappetizing foods (e.g., 100 g of raw potato) (Tacket et al., 2000).

4. Cost—The edible cell-based vaccines could incur a lower cost on producers, healthcare systems, and patients. Elimination of purification steps, less stringent storage and transportation requirements, and self-administration all result in lower costs. Moreover, there is already economic pressure for cultured meat to be cost-competitive with conventional products. As resources are developed for more cost-effective production of non-therapeutic cultured meat, these advances will 1 reduce the cost of vaccine-supplemented foods produced through similar methods.

Engineer muscle cells to produce hemagglutinin virus-like particles (HA VLPs). Muscle precursor cells isolated from Manduca sexta (caterpillar) and Bos taurus (cow) will be engineered to synthesize HA VLPs via transposon-mediated transgenesis. Insect and bovine cells will be proliferated and differentiated as described previously (Baryshyan et al., 2012; Simsa et al., 2019). We will evaluate growth kinetics and differentiation efficiency in non-engineered and engineered cells to determine the effect of HA VLP production on muscle cell behavior.

Characterize yield, stability, and efficacy of HA VLPs using in vi&o models. Yield, stability and efficacy of HA VLPs will be quantified for both cell types at three points: (a) after proliferation, (b) after differentiation, and (c) after simulated cooking. Stability and efficacy will be evaluated with the aid of three novel in vitro assays: (1) an in vitro simulated digestion to access VLP stability along the gastrointestinal tract, (2) an in vitro intestine model developed by our group to ensure VLP transportation across the mucosal barrier, and (3) an ALS assay to analyze VLP efficacy via ELISA for antibodies secreted by in vitro lymphocytes (Chang and Sack, 2001).

### Methods:

Muscle cell culture. Insect embryonic precursor cells (EPCs) will be isolated as described previously (Baryshyan et al., 2012). The cells will be isolated from Manduca sexta (tobacco hornworm) embryos and cultured in xenogen-free growth medium (Donaldson and Shuler, 1998) supplemented with methoprene. For differentiation experiments, medium will be supplemented with 1 μg/mL of the insect molting hormone 20-hydroxyecdysone (20-HEXRubio et al., 2019). Bovine muscle satellite cells will be isolated from the semitendinosus of a young beef cow as described previously (Simsa et al., 2019). Cells will be expanded on flasks coated with iMatrix recombinant laminin-511, fed basal medium supplemented with 20% fetal bovine serum and 1 ng/mL fibroblast growth factor 2, and differentiated in low-serum medium.

Transposon-mediated transgenesis. Influenza HA gene sequences from three viruses (i.e., H1N1, H3N2, and H5N1) will be codon optimized for bovine or insect cell expression, synthesized, and cloned into Sleeping Beauty transposon vectors under control of the constitutive promoters CMV and Ac5 for mammalian and insect expression, respectively (Kowarz et al., 2015; Shoji et al., 2008). A trivalent VLP will be generated by linking HA sequences with 2A ribosomal-skipping sequences to enable simultaneous expression of all HA fragments under a single promoter (Szymczak and Vignali, 2005). All genes will be linked via 2A sequences to genes for green fluorescent protein (GFP) and puromycin resistance. Cloned transposon vectors will be co-delivered to cells alongside a Sleeping Beauty transposase vector using a lipofection reagent protocol, as previously utilized by our lab (Stout et al.; Zayed et al., 2004). Stably transfected cells will be selected for with puromycin treatment and verified by detection of GFP expression.

Cell expansion. Insect cells will be incubated at 25° C. without carbon dioxide, medium will be refreshed once a week, and cells will be passaged at 90% confluency. Bovine cells will be incubated at 37° C. with 5% carbon dioxide, medium will be refreshed every other day, and cells will be passaged at 70% confluency. Growth kinetics will be analyzed via the CyQuant proliferation assay.

Cell differentiation. Cells will be differentiated using 20-HE supplementation (insect) or low-serum differentiation medium (bovine) for at least 7 days. Differentiation efficiency will be assessed by immunostaining for myosin heavy chain (MHC) and cell nuclei. MHC expression, myotube length, and fusion index will be quantified by Fiji image analysis.

Thermal treatment. To simulate cooking, differentiated tissues will be cooked in boiling water (100° C.) until they reach a temperature of 70° C. (i.e., the recommended temperature for cooking meat). Samples will be chilled at 4° C. for 24 hours before VLP analysis.

Western blot & SDS-PAGE. VLPs will be extracted from cell cultures and purified through ultracentrifugation and gradient iodixanol purification. These methods have been previously used to extract and analyze HA VLPs produced in insect and mammalian cell culture (Thompson et al., 2015). Purified samples will be loaded on a Tris-Glycine gel and run for 40 minutes at 200 V. The protein will be transferred to a nitrocellulose membrane, blocked, and incubated overnight with a primary anti-HA monoclonal antibody and an HRP-conjugated secondary antibody for 1 hour. Yield will be quantified via densitometric analysis. To analyze VLP size, purified VLPs (10 μg) will be loaded on a 12% SDS-PAGE gel and stained with Coomassie blue.

In vitro digestion. VLP cell cultures will be exposed to an in vitro digestion assay designed to mimic the dynamic conditions of the gastrointestinal tract. Cells will be incubated in buffers emulating the pH of the salivary (2 minutes at 37° C.), gastric (2 hours at 37° C.) and intestinal (2 hours at 37° C.) phases of digestion (Marseglia et al., 2019). Compositions are as follows: in vitro salivary fluid will be a 6.2 pH buffer supplemented with amylase, in vitro gastric fluid will be a 3.0 pH buffer supplemented with pepsin, and in vitro intestinal fluid will be a 7.0 pH buffer supplemented with pancreatin and bile solution. Upon digestion, stability of VLPs will be assessed via transmission electron microscopy, dynamic light scattering, and the following bioavailability and efficacy assays.

Transmission electron microscopy. Purified VLP samples will be diluted and placed on 200 Hex carbon coated and glow-discharged grids. Grids will be wasted and incubated with 1.5% uranyl formate. After drying, grids will be imaged with a transmission electron microscope.

Dynamic light scattering. Purified VLPs will be analyzed with a NanoBrook Zeta analyzer at a concentration of 500 μg/mL.

In vitro bioavailability assay. To study uptake in intestinal cells, we will employ a Caco-2 bioavailability assay. VLP-containing engineered tissue will be subjected to in vitro digestion as described above. VLP yield within the digested solution will be determined as described above. The digested solution will then be applied to a cell culture of Caco-2 cells. Caco-2 cells will be cultured on Transwell inserts, seeded at 500,000 cells/cm2, and fed RPMI 1640 medium supplemented with 10% fetal bovine serum and 1% antibiotic. Caco-2 cells will be treated with 5-150 μg of HA VLPs, cell viability will be quantified via MTT assay, and VLP uptake by Caco-2 cells will be measured by immunostaining (Lazorova et al.).

Vaccine efficacy assay. It has been reported that the induction of cytokine expression, in either primary human peripheral blood mononuclear cells (PMBCs) or the THP-1 line of human monocytic cells, may serve as a valid in vitro assay of vaccine efficacy (Desheva et al., 2019). We will employ a similar strategy. PMBCs or THP-1 cells will be cultured in 96-well plates. Preparations of digested VLP-expressing tissue will be generated as described above. PMBC or THP-1 cultures will be treated with serial dilutions of digested tissue at each stage of digestion. Purified VLPs will be used as a positive control. At several time points (4 hr, 24 hr, and 7 days after treatment), cell culture supernatant will be collected for ELISA of secreted cytokines, and cells will be collected and subjected to total RNA extraction for q-PCR analysis of cytokine mRNA expression levels. We will assess the cytokines IFNg, TNFa, IL-2, and IL-6. As purified VLPs have been used clinically as vaccines, the cytokine expression profile of purified VLP-treated cells will be used as our gold standard. The expression profiles generated from digested engineered meat-treated cells will be compared to this gold standard to determine if a similar immuno-stimulatory response can be achieved.

### REFERENCES

Ashok, A., Brison, M., and LeTallec, Y. (2017). Improving cold chain systems: Challenges and solutions. Vaccine 35, 2217-2223. doi:10.1016/j.vaccine.2016.08.045.
Baryshyan, A. L., Woods, W., Trimmer, B. A., and Kaplan, D. L. (2012). Isolation and Maintenance-Free Culture of Contractile Myotubes from Manduca sexta Embryos. PLoS One 7, e31598. doi:10.1371/journal.pone.0031598.
Bonanni, P., Picazo, J. J., and Remy, V. (2015). The intangible benefits of vaccination—what is the true economic value of vaccination? J. Mark. Access Heal. Policy 3, 26964. doi:10.3402/jmahp.v3.26964.
Centers for Disease Control and Prevention (2019). How Influenza (Flu) Vaccines Are Made. Seas. Influ. Available at: https://www.cdc.gov/flu/prevent/how-fluvaccine-made.htm [Accessed Feb. 15, 2020].
Centers for Disease Control and Prevention (2020). Disease Burden of Influenza. Seas. Influ. Available at: https://www.cdc.gov/flu/about/burden/rndex.html [Accessed Feb. 15, 2020].
Chang, H. S., and Sack, D. A. (2001). Development of a novel in vitro assay (ALS assay) for evaluation of vaccine-induced antibody secretion from circulating mucosal lymphocytes. Clin. Diagn. Lab. Immunol. 8, 482-488. doi:10.1128/CDLI.8.3.482-488.2001.
Chen, J. R., Liu, Y. M., Tseng, Y. C., and Ma, C. (2020). Better influenza vaccines: An industry perspective. J. Biomed. Sci. 27. doi:10.1186/s12929-020-0626-6.
Concha, C., Cañas, R., Macuer, J., Torres, M. J., Herrada, A. A., Jamett, F., et al. (2017). Disease Prevention: An Opportunity to Expand Edible Plant-Based Vaccines?doi:10.3390/vaccines5020014.
Cook, L. S. (2016). Needle phobia. J. Infus. Nurs. 39, 273-279. doi:10.1097/NAN.0000000000000184.
D'Aoust, M. A., Couture, M. M. J., Charland, N., Trepanier, S., Landry, N., Ors, F., et al. (2010). The production of hemagglutinin-based virus-like particles in plants: A rapid, efficient and safe response to pandemic influenza. Plant Biotechnol. J. 8, 607-619. doi:10.1111/j.1467-7652.2009.00496.x.
Datar, I., and Betti, M. (2010). Possibilities for an in vitro meat production system. Innov. Food Sci. Emerg. Technol. 11, 13-22. doi:10.1016/j.ifset.2009.10.007.
Desheva, Y. A., Leontieva, G. F., Kramskaya, T. A., Landgraf, G. O., Sychev, I. A., Rekstin, A. R., et al. (2019). Factors of early protective action of live influenza vaccine combined with recombinant bacterial polypeptides against homologous and heterologous influenza infection. Heliyon 5, e01154. doi:10.1016/j.heliyon.2019.e01154.
Donaldson, M. S., and Shuler, M. L. (1998). Low-Cost Serum-Free Medium for the BTI-Tn5B1-4 Insect Cell Line. Biotechnol. Prog. 14, 573-579. doi:10.1021/bp9800541.
Fraser, R. Z., Shitut, M., Agrawal, P., Mendes, O., and Klapholz, S. (2018). Safety Evaluation of Soy Leghemoglobin Protein Preparation Derived From Pichia pastoris, Intended for Use as a Flavor Catalyst in Plant-Based Meat. Int. J. Toxicol. 37, 241-262. doi:10.1177/1091581818766318.
Kowarz, E., Loscher, D., and Marschalek, R. (2015). Optimized Sleeping Beauty transposons rapidly generate stable transgenic cell lines. Biotechnol. J. 10, 647-653. doi:10.1002/biot.201400821.
Lazorova, L., Engstrom, A., and Sjijlander, A. Transport of an influenza virus vaccine formulation (iscom) in Caco-2 cells.
Marseglia, A., Dellafiora, L., Prandi, B., Lolli, V., Sforza, S., Cozzini, P., et al. (2019). Simulated gastrointestinal digestion of cocoa: Detection of resistant peptides and in silico/in vitro prediction of their ace inhibitory activity. Nutrients 11. doi:10.3390/nu11050985.
Mishra, N., Gupta, N., Khatri, K., Goyal, A. K., and Vyas, S. P. (2008). Edible vaccines: A new approach to oral immunization.
Ortiz, J. R., and Neuzil, K. M. (2019). Influenza Immunization in Low- and Middle-Income Countries: Preparing for Next-Generation Influenza Vaccines. J. Infect. Dis. 219, S97-S106. doi:10.1093/infdis/jiz024.
Post, M. J. (2012). Cultured Meat from Stem Cells: Challenges and Prospects. Meat Sci. 92, 297-301. doi:10.1016/j.meatsci.2012.04.008.
Rubio, N. R., Fish, K. D., Trimmer, B. A., and Kaplan, D. L. (2019). In Vitro Insect Muscle for Tissue Engineering Applications. ACS Biomater. Sci. Eng. 5, 1071-1082. doi:10.1021/acsbiomaterials.8b01261.
Schiele, J. T., Quinzler, R., Klimm, H. D., Pruszydlo, M. G., and Haefeli, W. E. (2013). Difficulties swallowing solid oral dosage forms in a general practice population: Prevalence, causes, and relationship to dosage forms. Eur. J. Clin. Pharmacol. 69, 937-948. doi:10.1007/s00228-012-1417-0.
Schneider-Ohrum, K., and Ross, T. M. (2012). Virus-like particles for antigen delivery at mucosal surfaces. Curr. Top. Microbiol. Immunol. 354, 53-73. doi:10.1007/82_2011_135.
Serradell, M. C., Rupil, L. L., Martino, R. A., Prucca, C. G., Carranza, P. G., Saura, A., et al. (2019). Efficient oral vaccination by bioengineering virus-like particles with protozoan surface proteins. Nat. Commun. 10, 361. doi:10.1038/s41467-018-08265-9.
Shoji, Y., Chichester, J. A., Bi, H., Musiychuk, K., de la Rosa, P., Goldschmidt, L., et al. (2008). Plant-expressed HA as a seasonal influenza vaccine candidate. Vaccine 26, 2930-2934. doi:10.1016/j.vaccine.2008.03.045.
Simsa, R., Yuen, J., Stout, A., Rubio, N., Fogelstrand, P., and Kaplan, D. L. (2019). Extracellular heme proteins influence bovine myosatellite cell proliferation and the color of cell-based meat. Foods 8. doi:10.3390/foods8100521.
Stout, A. J., Mirliani, A. B., Soule-Albridge, E. L., Cohen, J. M., and Kaplan, D. L. Engineering carotenoid production in mammalian cells for nutritionally enhanced cell-cultured foods. Rev.
Szymczak, A. L., and Vignali, D. A. (2005). Development of 2A peptide-based strategies in the design of multicistronic vectors. Expert Opin. Biol. Ther. 5, 627-638. doi:10.1517/14712598.5.5.627.
Tacket, C. O., Mason, H. S., Losonsky, G., Estes, M. K., Levine, M. M., and Arntzen, C. J. (2000). Human Immune Responses to a Novel Norwalk Virus Vaccine Delivered in Transgenic Potatoes. J. Infect. Dis. 182, 302-305. doi:10.1086/315653.
Thompson, C. M., Petiot, E., Mullick, A., Aucoin, M. G., Henry, O., and Kamen, A. A. (2015). Critical assessment of influenza VLP production in Sf9 and HEK293 expression systems. BMC Biotechnol. 15, 31. doi:10.1186/s12896-015-0152-x.
Vela Ramirez, J. E., Sharpe, L. A., and Peppas, N. A. (2017). Current State and Challenges in Developing Oral Vaccines. Adv. Drug Deliv. Rev. 114, 116-131. doi:10.1016/j.addr.2017.04.008.
World Health Organization (2015). World Health Statistics 2012. World Health Organization.
Young, E. (2018). 41 Percent of Americans Do Not Intend to Get a Flu Shot this Season. Natl. Opin. Res. Cent.
Zayed, H., Izsvik, Z., Walisko, O., and Ivics, Z. (2004). Development of Hyperactive Sleeping Beauty Transposon Vectors by Mutational Analysis. Mol. Ther. 9, 292-304. doi:10.1016/J.YMTHE.2003.11.024.

### Example 2—Antigens for Edible Vaccines Against Various Diseases

As is described in Example 1, edible cell-based vaccines against influenza can be created by engineering cells to express influenza antigens, such hemagglutinin (HA), neuraminidase (NA), matrix protein 1 (M1), and/or matrix protein 2 (M2). The amino acid sequences of HA subtypes 1-16 are provided as SEQ ID NOs:1-16, respectively; the amino acid sequences of NA subtypes 1-9 are provided as SEQ ID NOs:17-25, respectively; and the amino acid sequences of M1 and M2 are provided as SEQ ID NOs:26-27, respectively. Further, the amino acid sequences of the HA polypeptide from H1N1 (A/Hawaii/70/2019), H3N2 (A/Kansas/14/2017), and H5N1 (A/Egypt/2321-NAMRU3/2007) are provided as SEQ ID NOs:28-30, respectively.

Additionally, the edible cell-based vaccine platform described herein could also be used to generate vaccines against coronaviruses such as severe acute respiratory syndrome coronavirus (SARS-CoV), severe acute respiratory syndrome coronavirus 2 (SARS-CoV-2), and Middle East respiratory syndrome coronavirus (MERS-CoV). Such vaccines can be created by engineering cells to express coronavirus antigens, such as the spike (S), envelope (E), membrane (M), and/or receptor binding domain (RBD) proteins. The amino acid sequences of the S, E, and M proteins of SARS-CoV are provided as SEQ ID NOs:31-33, respectively; the amino acid sequences of the S, E, M, and RBD proteins of SARS-CoV-2 are provided as SEQ ID NOs:34-37, respectively; and the amino acid sequences of the S, E, and M proteins of MERS-CoV are provided as SEQ ID NOs:38-40, respectively.

The edible cell-based vaccine platform could also be used to generate vaccines against hepatitis B. Such vaccines can be created by engineering cells to express hepatitis B antigens, such major hepatitis B surface antigens (HBsAg), which are classified as large (L-HBsAg), middle (M-HBsAg), and small (S-HBsAg) proteins. The amino acid sequences of L-HBsAg, M-HBsAg, and S-HBsAg from hepatitis B are provided as SEQ ID NOs:41-43, respectively.

The edible cell-based vaccine platform could also be used to generate vaccines against human papillomavirus (HPV). Such vaccines can be created by engineering cells to express HPV antigens, such as L1. The amino acid sequences of the L1 protein from HPV type 6, 11, 16, and 18 are provided as SEQ ID NOs:44-47, respectively.

The edible cell-based vaccine platform could also be used to generate vaccines against human parvovirus. Such vaccines can be created by engineering cells to express parvovirus antigens such as viral protein 1 (VP1) and/or viral protein 2 (VP2). The amino acid sequences of VP1 and VP2 are provided as SEQ ID NOs:48 and 49, respectively.

The edible cell-based vaccine platform could also be used to generate vaccines against Norwalk virus. Such vaccines can be created by engineering cells to express Norwalk virus antigens such as capsid protein (CP). The amino acid sequence of CP is provided as SEQ ID NO: 50.

1. An engineered cell comprising a heterologous polynucleotide encoding a viral antigen, wherein the cell is a muscle cell, a muscle precursor cell, a fibroblast, or an adipocyte from a non-human mammal, an insect, a fish, or a bird.

2. The engineered cell of claim 1, wherein the viral antigen is an influenza antigen, a coronavirus antigen, a hepatitis B antigen, a human papilloma virus antigen, a parvovirus antigen, or a Norwalk virus antigen.

3. The engineered cell of claim 2, wherein the viral antigen comprises at least a portion of a protein selected from: an influenza hemagglutinin protein, an influenza neuraminidase protein, an influenza matrix protein, a coronavirus spike protein, a coronavirus envelope protein, a coronavirus membrane protein, a coronavirus receptor binding domain protein, a major hepatitis B surface antigen protein, a human papillomavirus L1 protein, a parvovirus viral protein, and a Norwalk virus capsid protein.

4. The engineered cell of claim 1, wherein the viral antigen has a sequence that is at least 90% identical to an amino acid sequence selected from SEQ ID NOs:1-50.

5. The engineered cell of claim 3, wherein the viral antigen comprises at least a portion of an influenza hemagglutinin protein from H₁N₁, H₃N₂, or H₅N₁.

6. The engineered cell of claim 1, wherein the cell comprising one or more heterologous polynucleotides encoding an influenza hemagglutinin protein, wherein the hemagglutinin protein is selected from the group consisting of H₁N₁, H₃N₂, and H₅N₁.

7. The engineered cell of claim 1, wherein the cell is a Pax7+ mammalian muscle precursor cell.

8. The engineered cell of claim 7, wherein the cell is a Pax7+ bovine satellite cell.

9. The engineered cell of claim 1, wherein the cell is a mammalian multinucleated myotube expressing myosin heavy chain (MHC) and actin.

10. The engineered cell of claim 1, wherein the cell is an insect muscle cell or insect muscle precursor cell.

11. The engineered cell of claim 10, wherein the insect muscle cell is a multinucleated myotube derived from a Drosophila adult muscle progenitor cell (DrAMPC) or a Manduca sexta embryonic precursor cell (Ms-EPC).

12. The engineered cell of claim 11, wherein the multinucleated myotube expresses myosin heavy chain and ecdysone receptor.

13. A cultured meat product comprising the engineered cell of claim 1 seeded on a food safe substrate.

14. The cultured meat product of claim 13, wherein the substrate is a film.

15. The cultured meat product of claim 13, wherein the substrate is a sponge or three-dimensional matrix.

16. The cultured meat product of claim 13, wherein the substrate is a chitosan substrate.

17. The cultured meat product of claim 13, wherein the product comprises a confluent culture of the engineered cells.

18. An edible vaccine comprising the cultured meat product comprising a heterologous polynucleotide encoding a viral antigen, wherein the cell is a muscle cell, a muscle precursor cell, a fibroblast, or an adipocyte from anon-human mammal, an insect, a fish, or a bird.

19. An edible vaccine comprising the engineered cell comprising a heterologous polynucleotide encoding a viral antigen, wherein the cell is a muscle cell, a muscle precursor cell, a fibroblast, or an adipocyte from a non-human mammal, an insect, a fish, or a bird.

20. A method for inducing an immune response against a virus in a subject comprising administering an effective amount of the edible vaccine to the subject, the edible vaccine comprising a heterologous polynucleotide encoding a viral antigen, wherein the cell is a muscle cell, a muscle precursor cell, a fibroblast, or an adipocyte from a non-human mammal, an insect, a fish, or a bird.

21. The method of claim 20, wherein the subject is a human.

22. The method of claim 20, further comprising administering the edible vaccine.


SEQUENCE LISTING (50 sequences)

SEQ ID NO: 1 - protein, 565 aa, Influenza A virus
  MISC_FEATURE (1)..(565): HA Type 1
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 2 - protein, 562 aa, Influenza A virus
  MISC_FEATURE (1)..(562): HA Type 2
MAIIYLILLFTAVRGDQICIGYHANNSTEMVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSDYPVAKGSYNNTSGEQMLIIWGVHHPNDETEQRTLYQNVGTYVSVGTSTLNKRSTPEIATRLKVNGQGGRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKTGTYDYPKYEEESKLNRNEIKGVKLSSMGVYQILAIYATVAGSLSLAIMMAGISFWMCSNGSLQCRICI

SEQ ID NO: 3 - protein, 566 aa, Influenza A virus
  MISC_FEATURE (1)..(566): HA Type 3
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIESRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 4 - protein, 564 aa, Influenza A virus
  MISC_FEATURE (1)..(564): HA Type 4
MLSITILFLLIAEGSSQNYTGNPVICLGHHAVPNGTMVKTLTDDQVEVVTAQELVESQHLPELCPSPLRLVDGQTCDIVNGALGSPGCDHLNGAEWDVFIERPTAVDTCYPFDVPDYQSLRSILANNGKFEFIAEEFQWNTVKQNGKSGACKRANVNDFFNRLNWLTKSDGNAYPLQNLTKVNNGDYARLYIWGVHHPSTDTEQTNLYENNPGRVTVSTQTSQTSVVPNIGSRPWVRGQSGRISFYWTIVEPGDLIVFNTIGNLIAPRGHYKLNSQKKSTILNTAVPIGSCVSKCHTDRGSISTTKPFQNISRISIGDCPKYVKQGSLKLATGMRNIPEKATRGLFGAIAGFIENGWQGLIDGWYGFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDKGNGCFEIFHQCDNSCIESIRNGTYDHDIYRDEAINNRFQIQGVKLTQGYKDIILWISFSISCFLLVALLLAFILWACQNGNIRCQICI

SEQ ID NO: 5 - protein, 568 aa, Influenza A virus
  MISC_FEATURE (1)..(568): HA Type 5
MEKTVLLLATVSLVKSDQICIGYHANNSTEQVDTIMEKNVTVTHAQDILERTHNGKLCDLNGVKPLILRDCSVAGWLLGNPMCDEFINVPEWSYIVEKASPANDLCYPGNFNDYEELKHLLSRINHFEKIQIIPKSSWSNHDASSGVSSACPYLGRSSFFRNVVWLIKKNSAYPTIKRSYNNTNQEDLLVLWGIHHPNDAAEQTKLYQNPTTYISVGTSTLNQRLVPEIATRPKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRRKKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESMGTYQILSIYSTVASSLALAIMVAGLSLWMCSNGSLQCRICI

SEQ ID NO: 6 - protein, 566 aa, Influenza A virus
  MISC_FEATURE (1)..(566): HA Type 6
MIAIIVIAILVAAGRSDKICIGYHANNSTTQVDTILEKNVTVTHSVELLENQKEERFCKILNKAPLDLRGCTIEGWILGNPQCDLLLGDQSWSYIVERPTAQNGICYPGVLNEVEELKALIGSGERVERFEMFPKSTWAGVDTSSGVTRACPYNTGSSFYRNLLWIIKTKSAAYPVIKGTYNNTGKQPILYFWGVHHPPDTNEQNTLYGSGDRYVRMGTESMNFAKSPEIAARPAVNGQRGRIDYYWSVLKPGETLNVESNGNLIAPWYAYRFVSTINKGAVFKSNLPIENCDATCQTIAGVLRTNKTFQNVSPLWIGECPKYVKSGSLRLATGLRNVPQIETRGLFGAIAGFIEGGWTGMIDGWYGYHHENSQGSGYAADRESTQKAIDGITNKVNSIIDKMNTQFEAVDHEFSNLERRIDNLNKRMEDGFLNVWTYNAELLVLLENERTLDLHDANVKNLYEKVKSQLRDNANDLGNGCFEFWHKCDNECIESVKNGTYDYPKYQDESKLNRQEIESVKLENLGVYQILAIYSTVSSSLVLVGLIIAMGLWMCSNGSMQCRICI

SEQ ID NO: 7 - protein, 560 aa, Influenza A virus
  MISC_FEATURE (1)..(560): HA Type 7
MNTQILVFALVAIIPTNADKICLGHHAVSNGTKVNTLTERGVEVVNATETVERTNVPRICSKGKRTVDLGQCGLLGTITGPPQCDQFLEFSADLIIERREGSDVCYPGKFVNEEALRQILRESGGIDKETMGFTYSGIRTNGATSACRRSGSSFYAEMKWLLSNTDNAAFPQMTKSYKNTRKDPALIIWGIHHSGSTTEQTKLYGSGNKLITVGSSNYQQSFVPSPEARPQVNGQSGRIDFHWLMLNPNDTVTFSFNGAFIAPDRASFLRGKSMGIQSGVQVDANCEGDCYHSGGTIISNLPFQNINSRAVGKCPRYVKQESLLLATGMKNVPEIPKGRGLFGAIAGFIENGWEGLIDGWYGFRHQNAQGEGTAADYKSTQSAIDQVTGKLNRLIEKTNQQFKLIDNEFTEVEKQIGNVINWTRDSMTEVWSYNAELLVAMENQHTIDLADSEMNKLYERVKRQLRENAEEDGTGCFEIFHKCDDDCMASIRNNTYDHSKYREEAMQNRIQIDPVKLSSGYKDVILWFSFGASCFILLAIAMGLVFICVKNGNMRCTICI

SEQ ID NO: 8 - protein, 565 aa, Influenza A virus
  MISC_FEATURE (1)..(565): HA Type 8
MEKFIAIATLASTNAYDRICIGYQSNNSTDTVNTLIEQNVPVTQTMELVETEKHPAYCNTDLGAPLELRDCKIEAVIYGNPKCDIHLKDQGWSYIVERPSAPEGMCYPGSVENLEELRFVFSSAASYKRIRLFDYSRWNVTRSGTSKACNASTGGQSFYRSINWLTKKEPDTYDFNEGAYVNNEDGDIIFLWGIHHPPDTKEQTTLYKNANTLSSVTTNTINRSFQPNIGPRPLVRGQQGRMDYYWGILKRGETLKIRTNGNLIAPEFGYLLKGESYGRIIQNEDIPIGNCNTKCQTYAGAINSSKPFQNASRHYMGECPKYVKKASLRLAVGLRNTPSVEPRGLFGAIAGFIEGGWSGMIDGWYGFHHSNSEGTGMAADQKSTQEAIDKITNKVNNIVDKMNREFEVVNHEFSEVEKRINMINDKIDDQIEDLWAYNAELLVLLENQKTLDEHDSNVKNLFDEVKRRLSANAIDAGNGCFDILHKCDNECMETIKNGTYDHKEYEEEAKLERSKINGVKLEENTTYKILSIYSTVAASLCLAILIAGGLILGMQNGSCRCMFCI

SEQ ID NO: 9 - protein, 560 aa, Influenza A virus
  MISC_FEATURE (1)..(560): HA Type 9
METKAIIAALLMVTAANADKICIGYQSTNSTETVDTLTESNVPVTHTKELLHTEHNGMLCATDLGHPLILDTCTIEGLIYGNPSCDILLGGKEWSYIVERSSAVNGMCYPGNVENLEELRSLFSSAKSYKRIQIFPDKTWNVTYSGTSRACSNSFYRSMRWLTHKSNSYPFQNAHYTNNERENILFMWGIHHPPTDTEQTDLYKNADTTTSVTTEDINRTFKPVIGPRPLVNGQQGRIDYYWSVLKPGQTLRIRSNGNLIAPWYGHVLTGESHGRILKTDLNNGNCVVQCQTEKGGLNTTLPFHNISKYAFGNCPKYVGVKSLKLPVGLRNVPAVSSRGLFGAIAGFIEGGWPGLVAGWYGFQHSNDQGVGMAADKGSTQKAIDKITSKVNNIIDKMNKQYEVIDHEFNELEARLNMINNKIDDQIQDIWAYNAELLVLLENQKTLDEHDANVNNLYNKVKRALGSNAVEDGNGCFELYHKCDDQCMETIRNGTYDRQKYQEESRLERQKIEGVKLESEGTYKILTIYSTVASSLVLAMGFAAFLFWAMSNGSCRCNICI

SEQ ID NO: 10 - protein, 561 aa, Influenza A virus
  MISC_FEATURE (1)..(561): HA Type 10
MYKVVVIIALLGAVKGLDRICLGHHAVANGTIVKTLTNEQEEVTNATETVESTNLNKLCMKGRSYKDLGNCHPVGMLIGTPVCDPHLTGTWDTLIERENAIAHCYPGATINEEALRQKIMESGGISKMSTGFTYGSSITSAGTTKACMRNGGDSFYAELKWLVSKTKGQNFPQTTNTYRNTDTAEHLIIWGIHHPSSTQEKNDLYGTQSLSISVESSTYQNNFVPVVGARPQVNGQSGRIDFHWTLVQPGDNITFSDNGGLIAPSRVSKLTGRDLGIQSEALIDNSCESKCFWRGGSINTKLPFQNLSPRTVGQCPKYVNQRSLLLATGMRNVPEVVQGRGLFGAIAGFIENGWEGMVDGWYGFRHQNAQGTGQAADYKSTQAAIDQITGKLNRLIEKTNTEFESIESEFSETEHQIGNVINWTKDSITDIWTYNAELLVAMENQHTIDMADSEMLNLYERVRKQLRQNAEEDGKGCFEIYHTCDDSCMESIRNNTYDHSQYREEALLNRLNINPVKLSSGYKDIILWFSFGESCFVLLAVVMGLVFFCLKNGNMRCTICI

SEQ ID NO: 11 - protein, 565 aa, Influenza A virus
  MISC_FEATURE (1)..(565): HA Type 11
MEKTLLFAAIFLCVKADEICIGYLSNNSTDKVDTIIENNVTVTSSVELVETEHTGSFCSINGKQPISLGDCSFAGWILGNPMCDELIGKTSWSYIVEKPNPTNGICYPGTLESEEELRLKFSGVLEFNKFEVFTSNGWGAVNSGVGVTAACKFGGSNSFFRNMVWLIHQSGTYPVIKRTFNNTKGRDVLIVWGIHHPATLTEHQDLYKKDSSYVAVGSETYNRRFTPEINTRPRVNGQAGRMTFYWKIVKPGESITFESNGAFLAPRYAFEIVSVGNGKLFRSELNIESCSTKCQTEIGGINTNKSFHNVHRNTIGDCPKYVNVKSLKLATGPRNVPAIASRGLFGAIAGFIEGGWPGLINGWYGFQHRNEEGTGIAADKESTQKAIDQITSKVNNIVDRMNTNFESVQHEFSEIEERINQLSKHVDDSVVDIWSYNAQLLVLLENEKTLDLHDSNVRNLHEKVRRMLKDNAKDEGNGCFTFYHKCDNKCIERVRNGTYDHKEFEEESKINRQEIEGVKLDSSGNVYKILSIYSCIASSLVLAALIMGFMFWACSNGSCRCTICI

SEQ ID NO: 12 - protein, 564 aa, Influenza A virus
  MISC_FEATURE (1)..(564): HA Type 12
MEKFIILSTVLAASFAYDKICIGYQTNNSTETVNTLSEQNVPVTQVEELVHRGIDPILCGTELGSPLVLDDCSLEGLILGNPKCDLYLNGREWSYIVERPKEMEGVCYPGSIENQEELRSLFSSIKKYERVKMFDFTKWNVTYTGTSKACNNTSNQGSFYRSMRWLTLKSGQFPVQTDEYKNTRDSDIVFTWAIHHPPTSDEQVKLYKNPDTLSSVTTVEINRSFKPNIGPRPLVRGQQGRMDYYWAVLKPGQTVKIQTNGNLIAPEYGHLITGKSHGRILKNNLPMGQCVTECQLNEGVMNTSKPFQNTSKHYIGKCPKYIPSGSLKLAIGLRNVPQVQDRGLFGAIAGFIEGGWPGLVAGWYGFQHQNAEGTGIAADRDSTQRAIDNMQNKLNNVIDKMNKQFEVVNHEFSEVESRINMINSKIDDQITDIWAYNAELLVLLENQKTLDEHDANVRNLHDRVRRVLRENAIDTGDGCFEILHKCDNNCMDTIRNGTYNHKEYEEESKIERQKVNGVKLEENSTYKILSIYSSVASSLVLLLMIIGGFIFGCQNGNVRCTFCI

SEQ ID NO: 13 - protein, 566 aa, Influenza A virus
  MISC_FEATURE (1)..(566): HA Type 13
MALNVIATLTLISVCVHADRICVGYLSTNSSERVDTLLENGVPVTSSIDLIETNHTGTYCSLNGVSPVHLGDCSFEGWIVGNPACTSNFGIREWSYLIEDPAAPHGLCYPGELNNNGELRHLFSGIRSFSRTELIPPTSWGEVLDGTTSACRDNTGTNSFYRNLVWFIKKNNRYPVISKTYNNTTGRDVLVLWGIHHPVSVDETKTLYVNSDPYTLVSTKSWSEKYKLETGVRPGYNGQRSWMKIYWSLIHPGEMITFESNGGFLAPRYGYIIEEYGKGRIFQSRIRMSRCNTKCQTSVGGINTNRTFQNIDKNALGDCPKYIKSGQLKLATGLRNVPAISNRGLFGAIAGFIEGGWPGLINGWYGFQHQNEQGTGIAADKESTQKAIDQITTKINNIIDKMNGNYDSIRGEFNQVEKRINMLADRIDDAVTDIWSYNAKLLVLLENDKTLDMHDANVKNLHEQVRRELKDNAIDEGNGCFELLHKCNDSCMETIRNGTYDHTEYAEESKLKRQEIDGIKLKSEDNVYKALSIYSCIASSVVLVGLILSFIMWACSSGNCRFNVCI

SEQ ID NO: 14 - protein, 568 aa, Influenza A virus
  MISC_FEATURE (1)..(568): HA Type 14
MIALILVALALSHTAYSQITNGTTGNPIICLGHHAVENGTSVKTLTDNHVEVVSAKELVETNHTDELCPSPLKLVDGQDCHLINGALGSPGCDRLQDTTWDVFIERPTAVDTCYPFDVPDYQSLRSILASSGSLEFIAEQFTWNGVKVDGSSSACLRGGRNSFFSRLNWLTKATNGNYGPINVTKENTGSYVRLYLWGVHHPSSDNEQTDLYKVATGRVTVSTRSDQISIVPNIGSRPRVRNQSGRISIYWTLVNPGDSIIFNSIGNLIAPRGHYKISKSTKSTVLKSDKRIGSCTSPCLTDKGSIQSDKPFQNVSRIAIGNCPKYVKQGSLMLATGMRNIPGKQAKGLFGAIAGFIENGWQGLIDGWYGFRHQNAEGTGTAADLKSTQAAIDQINGKLNRLIEKTNEKYHQIEKEFEQVEGRIQDLEKYVEDTKIDLWSYNAELLVALENQHTIDVTDSEMNKLFERVRRQLRENAEDQGNGCFEIFHQCDNNCIESIRNGTYDHNIYRDEAINNRIKINPVTLTMGYKDIILWISFSMSCFVFVALILGFVLWACQNGNIRCQICI

SEQ ID NO: 15 - protein, 570 aa, Influenza A virus
  MISC_FEATURE (1)..(570): HA Type 15
MNTQIIVILVLGLSMVKSDKICLGHHAVANGTKVNTLTERGVEVVNATETVEITGIDKVCTKGKKAVDLGSCGILGTIIGPPQCDLHLEFKADLIIERRNSSDICYPGRFTNEEALRQIIRESGGIDKESMGFRYSGIRTDGATSACKRTVSSFYSEMKWLSSSMNNQVFPQLNQTYRNTRKEPALIVWGVHHSSSLDEQNKLYGTGNKLITVGSSKYQQSFSPSPGARPKVNGQAGRIDFHWMLLDPGDTVTFTFNGAFIAPDRATFLRSNAPSGIEYNGKSLGIQSDAQIDESCEGECFYSGGTINSPLPFQNIDSRAVGKCPRYVKQSSLPLALGMKNVPEKIRTRGLFGAIAGFIENGWEGLIDGWYGFRHQNAQGQGTAADYKSTQAAIDQITGKLNRLIEKTNKQFELIDNEFTEVEQQIGNVINWTRDSLTEIWSYNAELLVAMENQHTIDLADSEMNKLYERVRRQLRENAEEDGTGCFEIFHRCDDQCMESIRNNTYNHTEYRQEALQNRIMINPVKLSSGYKDVILWFSFGASCVMLLAIAMGLIFMCVKNGNLRCTICI

SEQ ID NO: 16 - protein, 564 aa, Influenza A virus
  MISC_FEATURE (1)..(564): HA Type 16
MAKTLYLLVLIFVKYSRADKICIGYLSNNSTDTVDTLTENGVPVTSSIDLVETNHTGTYCSLNGVSPIHLGDCSFEGWIVGNPSCASNINIREWSYLIEDPNAPHKLCFPGEVDNNGELRHLFSGVNSFSRTELIPPSKWGDILEGTTASCQNRGANSFYRNLIWLVNKLNKYPVVKGEYNNTTGRDVLVLWGIHHPDTEATANKLYVNKNPYTLVSTKEWSRRYELEIGTRIGDGQRSWMKIYWHLMHPGERITFESSGGLLAPRYGYIIEKYGTGRIFQSGVRLAKCNTKCQTSMGGINTNKTFQNIERNALGDCPKYIKSGQLKLATGLRNVPSIVERGLFGAIAGFIEGGWPGLINGWYGFQHQNEQGTGIAADKTSTQKAINEITTKINNIIEKMNGNYDSIRGEFNQVEKRINMIADRVDDAVTDIWSYNAKLLVLIENDRTLDLHDANVRNLHEQIKRALKDNAIDEGDGCFSILHKCNDSCMETIRNGTYNHEDYKEESQLKRQEIEGIKLKTEDNVYKILSIYSCIASSVVLVGLILAFILWACSSGNCRFNVCI

SEQ ID NO: 17 - protein, 469 aa, Influenza A virus
  MISC_FEATURE (1)..(469): NA Type 1
MNPNQKIITIGSICMVVGIISLILQIGNIISIWVSHSIQTGNQNHPETCNQSIITYENNTWVNQTYVNISNTNVVAGQDATSVILTGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGMGWLTIGISGPDNGAVAVLKYNGIITDTIKSWRNNILRTQESECACVNGSCFTIMTDGPSNGQASYKILKIEKGKVTKSIELNAPNYHYEECSCYPDTGKVMCVCRDNWHGSNRPWVSFDQNLDYQIGYICSGVFGDNPRPNDGTGSCGPVSSNGANGIKGFSFRYDNGVWIGRTKSTSSRSGFEMIWDPNGWTETDSSFSVRQDIVAITDWSGYSGSFVQHPELTGLDCMRPCFWVELIRGQPKENTIWTSGSSISFCGVNSDTVGWSWPDGAELPFSIDK

SEQ ID NO: 18 - protein, 451 aa, Influenza A virus
  MISC_FEATURE (1)..(451): NA Type 2
MNPNQKIITIGSVSLTIATVCFLMQIAILATTVTLHFKQNECSIPAKNQVVPCEPTIAERPKAVDYRIWSKPQCQITGFAPFSKDNSIRLSAGGDIWVTREPYVSCETSKCYQFALGQGTTLDNKHSNGTIHDRIPHRTLLMSELGVPFHLGTKQVCIAWSSSSCHDGKAWLHVCVTGDDRNATASFIYDGMLVDSISSWSQNILRTQESECVCINGTCTVVMTDGSASGKADTRILFIKEGKIAHISPLSGSAQHIEECSCYPQYSNVRCVCRDNWKGSNRPIIDINMADYSIDSSYVCSGLVGDTPRNDDSSSSSNCRDPNNERGNPGVKGWAFDDGNDVWMGRTISKDSRSGYETFKVIDGWATANSKSQTNRQVIVDNNNWSGYSGIFSVENKGCINRCFYVELIRGRPQETRVWWTSNSIVVFCGTSGTYGTGSWPDGANIDFMPI

SEQ ID NO: 19 - protein, 469 aa, Influenza A virus
  MISC_FEATURE (1)..(469): NA Type 3
MNPNQKIITIGVVNTTLSTIALLIGVGNLIFNTVIHEKIGDHQTVVYPTITAPVVPNCSDTIITYNNTVINNITTTIITEAERHFKPSLPLCPFRGFFPFHKDNAIRLGENKDVIVTREPYVSCDNDNCWSFALAQGALLGTKHSNGTIKDRTPYRSLIRFPIGTAPVLGNYKEICVAWSSSSCFDGKEWMHVCMTGNDNDASAQIIYAGKMTDSIKSWRRDILRTQESECQCIDGTCVVAVTDGPAANSADHRVYWIREGKVIKYENVPKTKIQHLEECSCYVDTDVYCVCRDNWKGSNRPWMRINNETILETGYVCSKFHSDTPRPADPSTVSCDSPSNVNGGPGVKGFGFKTGDDVWLGRTVSTNGRSGFEIIKVTEGWINSPNHAKSVTQTLVSNNDWSGYSGSFIVENNGCFQPCFYIELIRGRTNKNDDVSWTSNSIVTFCGLDNEPGSGNWPDGSNIGFMPK

SEQ ID NO: 20 - protein, 470 aa, Influenza A virus
  MISC_FEATURE (1)..(470): NA Type 4
MNPNQKIITIGSASIVLTTIGLLLQITSLCSIWFSHYNQVTQPHEQACSNNTTNYYNETFVNVTNVQNNYTTIIEPSAPNVVHYSSGRDLCPVKGWAPLSKDNGIRIGSRGEVFVIREPFISCSISECRTFFLTQGALLNDKHSNGTVKDRSPFRTLMSCPMGVAPSPSNSRFESVAWSATACSDGPGWLTLGITGPDATAVAVLKYNGIITDTLKSWKGNIMRTQESECVCQDEFCYTLITDGPSNAQAFYKILKIRKGKIVSVKDVNATGFHFEECSCYPSGTDVECVCRDNWRGSNRPWIRFNSDLDYQIGYVCSGIFGDNPRPVDGIGSCNSPVNNGKGRYGVKGFSFRYGDGVWIGRTKSLESRSGFEMVWDANGWVSTDKDSNGVQDIIDNNNWSGYSGSFSIRWETTGRNCTVPCFWVEMIRGQPKEKTIWTSGSSIAFCGVNSDTTGWSWPDGALLPFDIDK

SEQ ID NO: 21 - protein, 468 aa, Influenza A virus
  MISC_FEATURE (1)..(468): NA Type 5
MNPNQKIITIGSASLGLVIFNILLHVASITLGIISVTKDNKVHICNTTEVYNETVRVETVVIPVNNTIYLNHEPEFLNNTEPLCDVSGFAIVSKDNGIRIGSRGHIFVIREPFVSCGPSECRTFFLTQGALLNDKHSNNTVKDRSPYRALMSVPLGSSPNAYQAKFESVGWSATACHDGKKWMAIGVSGADDDAYAVIHYGGVPTDVIRSWRKQILRTQESSCVCIKGECYWVMTDGPANNQASYKIFKSQKGMVVDEKEISFQGGHIEECSCYPNMGKVECVCRDNWNGMNRPILIFDEKLEYEVGYLCAGIPTDTPRVQDSSFTGSCTNAVGRSGTNNYGVKGFGFRQGNSVWAGRTISVSSRSGFEVLLIEDGWIRPSKTISKKVEVLNNKNWSGYSGAFTIPTAMTSKNCIVPCFWLEMIRGKPEERTSIWTSSSSTVFCGVSSEVPGWSWDDGAILPFDIDKM

SEQ ID NO: 22 - protein, 470 aa, Influenza A virus
  MISC_FEATURE (1)..(470): NA Type 6
MNPNQKIICISATGMTLSVVSLLVGIANLGLNIGLHYKVGDTPNVNIPNVNGTNSTTTIINNNTQNNFTNITNIIQSKGGERTFLNLTKPLCEVNSWHILSKDNAIRIGEDAHILVTREPYLSCDPQGCRMFALSQGTTLRGRHANGTIHDRSPFRALISWEMGQAPSPYNTRVECIGWSSTSCHDGMSRMSICMSGPNNNASAVVWYGGRPITEIPSWAGNILRTQESECVCHKGVCPVVMTDGPANNRAATKIIYFKEGKIQKIEELAGNAQHIEECSCYGAGGVIKCICRDNWKGANRPVITIDPEMMTHTSKYLCSKVLTDTSRPNDPTNGNCDAPITGGSPDPGVKGFAFLDGENSWLGRTISKDSRSGYEMLKVPNAETDIQSGPISNQVIVNNQNWSGYSGAFIDYWANKECFNPCFYVELIRGRPKESSVLWTSNSIVALCGSKKRLGSWSWHDGAEIIYFE

SEQ ID NO: 23 - protein, 471 aa, Influenza A virus
  MISC_FEATURE (1)..(471): NA Type 7
MNPNQKLFALSGVAIALSVLNLLIGISNVGLNVSLHLKGEGVKQENNLTCTTITQNNTTVVENTYVNNTTIINKGTNLKAPNYLLLNKSLCSVEGWVVIAKDNAIRFGESEQIIVTREPYVSCDPSGCKMYALHQGTTIRNKHSNGTIHDRTTFRGLISTPLGTPPTVSNSDFICVGWSSTSCHDGVGRMTICIQGNNDNATATVYYNRRLTTTIKTWAKNILRTQESECVCYNGTCAVVMTDGPASSQAYTKIMYFHKGLIIKEEPLRGSARHIEECSCYGHDQKVSCVCRDNWQGANRPIIEIDMSTLEHTSRCVCTGVLTDTSRPGDKPNGDCSNPITGSPGAPGVKGFGFLNGDNTWLGRTISPRSRSGFEMLKIPNAETDPNSRIIERQEIVDNSNWSGYSGSFIDCWDEANECYNPCFYVELIRGRPEEAKYVWWTSNSLIALCGSPVSVGSGSFPDGAQIQYFS

SEQ ID NO: 24 - protein, 470 aa, Influenza A virus
  MISC_FEATURE (1)..(470): NA Type 8
MNPNQKIITIGSISLGLVVFNVLLHVVSIIVTVLVLGKGGNNGICNETVVREYNETVRIEKVTQWHNTNVVEYVPYWNGGTYMNNTEAICDAKGFAPFSKDNGIRIGSRGHIFVIREPFVSCSPIECRTFFLTQGSLLNDKHSNGTVKDRSPFRTLMSVEVGQSPNVYQARFEAVAWSATACHDGKKWMTVGVTGPDSKAVAVIHYGGVPTDVVNSWAGDILRTQESSCTCIQGDCYWVMTDGPANRQAQYRIYKANQGRIIGQTDISFNGGHIEECSCYPNDGKVECVCRDGWTGTNRPVLVISPDLSYRVGYLCAGIPSDTPRGEDTQFTGSCTSPMGNQGYGVKGFGFRQGTDVWMGRTISRTSRSGFEILRIKNGWTQTSKEQIRKQVVVDNLNWSGYSGSFTLPVELSGKDCLVPCFWVEMIRGKPEEKTIWTSSSSIVMCGVDYEVADWSWHDGAILPFDIDKM

SEQ ID NO: 25 - protein, 470 aa, Influenza A virus
  MISC_FEATURE (1)..(470): NA Type 9
MNPNQKILCTSATALVIGTIAVLIGITNLGLNIGLHLKPSCNCSHSQPEATNASQTIINNYYNDTNITQISNTNIQVEERAIRDFNNLTKGLCTINSWHIYGKDNAVRIGEDSDVLVTREPYVSCDPDECRFYALSQGTTIRGKHSNGTIHDRSQYRALISWPLSSPPTVYNSRVECIGWSSTSCHDGKTRMSICISGPNNNASAVIWYNRRPVTEINTWARNILRTQESECVCHNGVCPVVFTDGSATGPAETRIYYFKEGKILKWEPLAGTAKHIEECSCYGERAEITCTCRDNWQGSNRPVIRIDPVAMTHTSQYICSPVLTDNPRPNDPTVGKCNDPYPGNNNNGVKGFSYLDGVNTWLGRTISIASRSGYEMLKVPNALTDDKSKPTQGQTIVLNTDWSGYSGSFMDYWAEGECYRACFYVELIRGRPKEDKVWWTSNSIVSMCSSTEFLGQWDWPDGAKIEYFL

SEQ ID NO: 26 - protein, 252 aa, Influenza A virus
  MISC_FEATURE (1)..(252): M1
MSLLTEVETYVLSIIPSGPLKAEIAQKLEDVFAGKNTDLEALMEWLKTRPILSPLTKGILGFVFTLTVPSERGLQRRRFVQNALNGNGDPNNMDRAVKLYKKLKREITFHGAKEVALSYSTGALASCMGLIYNRMGTVTTEVAFGLVCATCEQIADSQHRSHRQMATTTNPLIRHENRMVLASTTAKAMEQMAGSSEQAAEAMEIANQARQMVQAMRTIGTHPNSSAGLRDNLLENLQAYQKRMGVQMQRFK

SEQ ID NO: 27 - protein, 97 aa, Influenza A virus
  MISC_FEATURE (1)..(97): M2
MSLLTEVETPTRTEWECRCSDSSDPLVVAANIIGILHLILWILDRLFFKCIYRRLKYGLKIGPSTEGVPESMREEYRQEQQSAVDVDDGHFVNIELE

SEQ ID NO: 28 - protein, 566 aa, Influenza A virus
  MISC_FEATURE (1)..(566): HA from H1N1 (A/Hawaii/70/2019)
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 29 - protein, 566 aa, Influenza A virus
  MISC_FEATURE (1)..(566): HA from H3N2 (A/Kansas/14/2017)
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 30 - protein, 568 aa, Influenza A virus
  MISC_FEATURE (1)..(568): HA from H5N1 (A/Egypt/2321-NAMRU3/2007(H5N1)
MEKIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGDFNDYEELKHLLSRINHFEKIQIIPKSSWSDYEASSGVSSACPYQGRSSFFRNVVWLIKKNNAYPTIKRSYNNTNQEDLLVLWGIHHPNDAAEQIRLYQNPTTYISVGTSTLNQRLVPKIATRSKVNGQSGRMEFFWTILKSNDAINFESNGNFIAPEYAYKIVKKGDSTIMKSELEYGNCNTKCQTPIGAINSSMPFHNIHPLTIGECPKYVKSNRLVLATGLRNSPQGERRRRKRGLFGAIAGFIEGGWQGMVDGWYGYHHSNEQGSGYAADKESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGIYQILSIYSTVASSLALAIMVAGLFLWMCSNGSLQCRICI

SEQ ID NO: 31 - protein, 1255 aa, SARS-CoV
  MISC_FEATURE (1)..(1255): S protein
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 32 - protein, 76 aa, SARS-CoV
  MISC_FEATURE (1)..(76): E protein
MYSFVSEETGTLIVNSVLLFFAFVVFLLVTLAILTALRLCAYCCNIVNVSLVKPTVYVYSRVKNLNSSEGVPDLLV

SEQ ID NO: 33 - protein, 221 aa, SARS-CoV
  MISC_FEATURE (1)..(221): M protein
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 34 - protein, 1273 aa, SARS-CoV-2
  MISC_FEATURE (1)..(1273): S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 35 - protein, 76 aa, SARS-CoV-2
  MISC_FEATURE (1)..(76): E protein
MYSFVSEETGTLIVNSVLLFLAFVVFLLVTLAILTALRLCAYCCNIVNVSLVKPTVYVYSRVKNLNSSEGVPDLLV

SEQ ID NO: 36 - protein, 222 aa, SARS-CoV-2
  MISC_FEATURE (1)..(222): M protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 37 - protein, 223 aa, SARS-CoV-2
  MISC_FEATURE (1)..(223): RBD protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 38 - protein, 1353 aa, MERS-CoV
  MISC_FEATURE (1)..(1353): S protein
MIHSVFLLMFLLTPTESYVDVGPDSAKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSAIIRKIYPAFMLGSSVGNFSYGKMGRFFNHTLVLLPDGCGTLLRAFYCILEPRSGNYCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLFGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVLQLVNANQYSPCVSIVPSTVWEDGDYYRKQLSPLEGGGWLVASGSTVAMTEQLQMGFGITVQYGTDTNSVCPKLEFANDTKIVSQLGNCVEYSLYGVSGRGVFQNCTAVGVPQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFESVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDSANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLSFIAGLVALALCVFFILCCTGCGTNCMGKLKCNRCCDRYEEYDLEPHKVHVH

SEQ ID NO: 39 - protein, 82 aa, MERS-CoV
  MISC_FEATURE (1)..(82): E protein
MLPFVQERIGLFIVNFFIFTVVCAITLLVCMAFLTATRLCVQCMTGFNTLLVQPALYLYNTGRLVYVKFQDSKPPLPPDEWV

SEQ ID NO: 40 - protein, 219 aa, MERS-CoV
  MISC_FEATURE (1)..(219): M protein
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 41 - protein, 389 aa, Hepatitis B
  MISC_FEATURE (1)..(389): L-HBsAg
MGTNLSVPNPLGFFPDHQLDPAFGANSNNPDWDFNPIKDHWPEANQVGVGAFGPGFTPPHGGALGWSPQAQGILTTVPKVPPPASTNRQSGRQPTPISPPLRDSHPQAMQWNSTTFHQALQDPRVRGLYFPAGGSSSGTLNPVQNIASHISSISSRTGDPVPNMENITSGFLGPLLVLQAGFFSLTKILTIPQSLDSWWTSLNFLGEAPVCHGQNSQSPTSNHSPTSCPPICPGYRWMCLRRFIIFLFILLLCLIFLLVLLDYQGMLPVCPLIPGTTTTSTGPCKTCTTPAQGNSMFPSCCCTKPTDGNCTCIPIPSSWAFAKYLWEWASVRFSWLSLLVPFVQWFAGLSPTVWLSVIWMMWYWGPSLYNILSPFIPLLPIFFCLWVYS

SEQ ID NO: 42 - protein, 281 aa, Hepatitis B
  MISC_FEATURE (1)..(281): M-HBsAg
MQWNSTTFHQTLQDPRVRALYFPAGGSSSGTVSPAQNTVSAISSILSKTGDPVPNMENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDGNCTCIPIPSSWAFAKYLWEWASVRFSWLSLLVPFVQWFVGLSPTVWLSVIWMMWFWGPSLYNILSPFMPLLPIFFCLWVYI

SEQ ID NO: 43 - protein, 226 aa, Hepatitis B
  MISC_FEATURE (1)..(226): S-HBsAg
MDNITSGLLGPLLVLQAVSFLLIKILTIPQSLDSWWTSLNFLGELPGCPGQNSQSPTSNHLPTSCPPTCPGYRWMCLRRFIIFLFILLLCLIFLLVLLDYQGMLPVCPLLPGSTTTSTGPCKTCTTLAQGTSMFPSCCCTKPSDGNCTCIPIPSSWALGKYLWEWASARFSWLSLLVQFVQWCVGLSPTVWLLVIWMIWYWGPNLCSILSPFIPLLPIFCYLWVSI

SEQ ID NO: 44 - protein, 500 aa, Human papillomavirus type 6
  MISC_FEATURE (1)..(500): L1
MWRPSDSTVYVPPPNPVSKVVATDAYVTRTNIFYHASSSRLLAVGHPYFSIKRANKTVVPKVSGYQYRVFKVVLPDPNKFALPDSSLFDPTTQRLVWACTGLEVGRGQPLGVGVSGHPFLNKYDDVENSGSGGNPGQDNRVNVGMDYKQTQLCMVGCAPPLGEHWGKGKQCTNTPVQAGDCPPLELITSVIQDGDMVDTGFGAMNFADLQTNKSDVPIDICGTTCKYPDYLQMAADPYGDRLFFFLRKEQMFARHFFNRAGEVGEPVPDTLIIKGSGNRTSVGSSIYVNTPSGSLVSSEAQLFNKPYWLQKAQGHNNGICWGNQLFVTVVDTTRSTNMTLCASVATSSTYTNSDYKEYMRHVEEYDLQFIFQLCSITLSAEVMAYIHTMNPSVLEDWNFGLSPPPNGTLEDTYRYVQSQAITCQKPTPEKEKPDPYKNLSFWEVNLKEKFSSELDQYPLGRKFLLQSGYRGRSSIRTGVKRPAVSKASAAPKRKRAKTKR

SEQ ID NO: 45 - protein, 501 aa, Human papillomavirus type 11
  MISC_FEATURE (1)..(501): L1
MWRPSDSTVYVPPPNPVSKVVATDAYVKRTNIFYHASSSRLLAVGHPYYSIKKVNKTVVPKVSGYQYRVFKVVLPDPNKFALPDSSLFDPTTQRLVWACTGLEVGRGQPLGVGVSGHPLLNKYDDVENSGGYGGNPGQDNRVNVGMDYKQTQLCMVGCAPPLGEHWGKGTQCSNTSVQNGDCPPLELITSVIQDGDMVDTGFGAMNFADLQTNKSDVPLDICGTVCKYPDYLQMAADPYGDRLFFYLRKEQMFARHFFNRAGTVGEPVPDDLLVKGGNNRSSVASSIYVHTPSGSLVSSEAQLFNKPYWLQKAQGHNNGICWGNHLFVTVVDTTRSTNMTLCASVSKSATYTNSDYKEYMRHVEEFDLQFIFQLCSITLSAEVMAYIHTMNPSVLEDWNFGLSPPPNGTLEDTYRYVQSQAITCQKPTPEKEKQDPYKDMSFWEVNLKEKFSSELDQFPLGRKFLLQSGYRGRTSARTGIKRPAVSKPSTAPKRKRTKTKK

SEQ ID NO: 46 - protein, 505 aa, Human papillomavirus type 16
  MISC_FEATURE (1)..(505): L1
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGAVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKL

SEQ ID NO: 47 - protein, 568 aa, Human papillomavirus type 18
  MISC_FEATURE (1)..(568): L1
MCLYTRVLILHYHLLPLYGPLYHPQPLPLHNILVYMVHIIICGHYIILFLRNVNVFPIFLQMALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 48 - protein, 781 aa, Human parvovirus B19
  MISC_FEATURE (1)..(781): VP1
MSKESGKWWESDDKFAKAVYQQFVEFYEKLTGTDLELIQILKDHYNISLDNPLENPSSLFDLVARIKNNLKNSPDLYSHHFQSHGQLSDHPHALSSSNSHAEPRGENAVLSSEDLHKPGQVSVQLPGTNYVGPGNELQAGPPQSAVDSAARIHDFRYSQLAKLGINPYTHWTVADEELLENIKNETGFQAQVVKDYFTLKGAAAPVAHFQGSLPEVPAYNASEKYPSMTSVNSAEASTGAGGGGSNSVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSSTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPPSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 49 - protein, 554 aa, Human parvovirus B19
  MISC_FEATURE (1)..(554): VP2
MTSVNSAEAITGAGGGGSNSAKSMWSEGATFTANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSSTGRLCMLVDHEYKYPYVLGQGQDTLAPELPIWVYFPPQYAYLSVGDVNTQGITGDSKKLASEESAFYVLEHSSFQLLGTGGSASMSYKFPAVPPENLEGCSQHFYEMYNPLYGSRLGVPDTLGGDPKFRSLTHEDHAIQPQNFMPRPLVNSVSTNEGDSSNTGAGKALTGLRKGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTIKLGPRKATGRWNPQPGVYPPHAAGHLPYGLYDPTATDAKQHHRHGYEKPEELWTAKSRVHPL

SEQ ID NO: 50 - protein, 530 aa, Norwalk virus
  MISC_FEATURE (1)..(530): CP
MMMASKDATSSVDGASASVQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSQQLTIAQATLFPHVIADVRTLDPIEVPLEDVRNVLFHNNDRNQQTMRLVCMLYTPLSTGGGTGDSFVVAGRVMTCPSPDFNFLFLVPPTVEQKTRPFTLPNLPLSSLSNSRAPLPISGMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTSVPHLGSIQANGIGSGNYIGVLSWVSPPSHPSGSQVDLWKIPNYGSSITEATHLAPSVYSPGFGEVLVFFMSKIPGPGGDSLPCLLPQGYISHLASEQAPTVGEGPLLHYVDPDTDRNLGEFKAYPDGFLTCVPNGASSGPQQLPINGVFVFVSWVSRFYQLKPVGTASTARGRLGLRR